(19) United States
(12) Patent Application Publication
(10) Pub. No.: US 2005/0228270 A1
(43) Pub. Date: Oct. 13, 2005

(54) Method and system for geometric distortion free tracking of 3-dimensional objects from 2-dimensional measurements

(76) Inventors: Charles Frederick Lloyd, Reading, MA (US); Michel Grimaud, Boston, MA (US); Alex Dun Li, Lebanon, NH (US); Lewis J. Levine, Weston, MA (US); Jon Thomas Lea, Hampstead, NH (US)

Correspondence Address: MCANDREWS HELD & MALLOY, LTD, 500 WEST MADISON STREET, SUITE 3400, CHICAGO, IL, 60661, US

(21) Appl. No.: 10/962,019

(22) Filed: Oct. 8, 2004

Related U.S. Application Data
(63) Continuation-in-part of application No. 10/817,652, filed on Apr. 2, 2004.

(51) Int. Cl. A61B 5/05

(52) U.S. Cl. 600/424; 600/407; 600/425

(57) ABSTRACT

Certain embodiments of the present invention provide a method and system for improved tracking of an internal object, such as an implant or part of the anatomy, during an image-guided operation. In an embodiment, the method may include obtaining a plurality of fiducials for an internal object from a plurality of projection views, obtaining a plurality of measurements for the internal object using the plurality of fiducials, forming a three-dimensional model of the internal object using the plurality of measurements from a plurality of projection views for use in tracking the internal object, and performing an image-guided operation using an image data set and a three-dimensional representation of the internal object. The fiducials may be indentations, curves, grooves and/or identifying features in the internal object. In an embodiment, the representation of the internal object is compared to a computer-generated model of the internal object.

### RELATED APPLICATIONS

The present application relates to, and claims priority from, as a continuation-in-part, U.S. application Ser. No. 10/817,652 filed on Apr. 1, 2004, and entitled “Method and System for Calibrating Deformed Instruments” (Attorney Docket Number 144382NV (15427US01)).

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to image-guided navigation. In particular, the present invention relates to a system and method for dynamic calibration of instruments, implants, or anatomy used in image-guided surgery and other tracking operations.

Medical practitioners, such as doctors, surgeons, and other medical professionals, often rely upon technology when performing a medical procedure, such as image-guided surgery or examination. A tracking system may provide positioning information for the medical instrument with respect to the patient or a reference coordinate system, for example. A medical practitioner may refer to the tracking system to ascertain the position of the medical instrument when the instrument is not within the practitioner's line of sight. A tracking system may also aid in pre-surgical planning.

The tracking or navigation system allows the medical practitioner to visualize the patient's anatomy and track the position and orientation of the instrument. The medical practitioner may use the tracking system to determine when the instrument is positioned in a desired location. The medical practitioner may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may also be used to track the position of items other than medical instruments in a variety of applications. That is, a tracking system may be used in other settings where the position of an instrument in an object or an environment is difficult to accurately determine by visual inspection. For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. The response signal is detected by a receiver. The signal emitted by the transponder may then be used to monitor the position of a person or object in a simulated environment.

Tracking systems may be ultrasound, inertial position, or electromagnetic tracking systems, for example. Electromagnetic tracking systems may employ coils as receivers and transmitters. Typically, an electromagnetic tracking system is configured in an industry-standard coil architecture (ISCA). ISCA uses three colocated orthogonal quasi-dipole transmitter coils and three colocated quasi-dipole receiver coils. Other systems may use three large, non-dipole, non-colocated transmitter coils with three colocated quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space.

The ISCA tracker architecture uses a three-axis dipole coil transmitter and a three-axis dipole coil receiver. Each three-axis transmitter or receiver is built so that the three coils exhibit the same effective area, are oriented orthogonally to one another, and are centered at the same point. If the coils are small enough compared to a distance between the transmitter and receiver, then the coil may exhibit dipole behavior. Magnetic fields generated by the trio of transmitter coils may be detected by the trio of receiver coils. Using three approximately concentrically positioned transmitter coils and three approximately concentrically positioned receiver coils, for example, nine parameter measurements may be obtained. From the nine parameter measurements and one known position or orientation parameter, a position and orientation calculation may determine position and orientation information for each of the transmitter coils with respect to the receiver coil trio with three degrees of freedom.

In medical and surgical imaging, such as intraoperative or perioperative imaging, images are formed of a region of a patient's body. The images are used to aid in an ongoing procedure with a surgical tool or instrument applied to the patient and tracked in relation to a reference coordinate system formed from the images. Image-guided surgery is of a special utility in surgical procedures such as brain surgery and arthroscopic procedures on the knee, wrist, shoulder or spine, as well as certain types of angiography, cardiac procedures, interventional radiology and biopsies in which x-ray images may be taken to display, correct the position of, or otherwise navigate a tool or instrument involved in the procedure.

Several areas of surgery involve very precise planning and control for placement of an elongated probe or other article in tissue or bone that is internal or difficult to view directly. In particular, for brain surgery, stereotactic frames that define an entry point, probe angle and probe depth are used to access a site in the brain, generally in conjunction with previously compiled three-dimensional diagnostic images, such as MRI, PET or CT scan images, which provide accurate tissue images. For placement of pedicle screws in the spine, where visual and fluoroscopic imaging directions may not capture an axial view to center a profile of an insertion path in bone, such systems have also been useful.

Many medical procedures involve a medical instrument, such as a drill, a catheter, scalpel, reducer rod, scope, shunt or other tool. Several mechanical instruments used in image-guided surgery are subjected to repeated high stress and become deformed over time as a result of high stress encountered during surgery or between uses. Additionally, many mechanical instruments used in image-guided surgery are manufactured with large error tolerances which may introduce unacceptable distortion or error in surgical navigation. Additionally, implants and anatomy may undergo deformations or changes in position with respect to an instrument during surgery. Distortions in a tracking system may cause the tracking system to be inaccurate. Simple calibration methods are currently unable to account for such deformations. Thus, many tools become unusable. A system and method that allows continued effective use of deformed or nominal parts would be highly desirable.

During image-guided surgery, internal objects—implants or parts of an anatomy—may change location, orientation and even shape. Changes in an implant or anatomy may interfere with an image-guided surgery. Given the three-dimensional nature of the internal objects, a single image often fails to show all the pertinent information for the object. To properly track the changes during surgery more images of the implant or anatomy are typically obtained, increasing the length and cost of the surgery. A system and method that reduces the number of images typically obtained to determine location, orientation and size would be highly desirable.

Some current systems use a one-point test to calibrate a mechanical instrument for surgical navigation. A tracker may be attached to the back end of the instrument. The front end of the instrument is placed in a dimple. If position of the tracker matches the predicted position, then the instrument is calibrated. If the instrument is deformed and does not calibrate, the instrument is bent until the instrument is capable of being calibrated or the instrument is discarded.

Electromagnetic tracking allows medical practitioners, such as a surgeon, to perform operations without a direct line of sight. Surgeons rely on electromagnetic trackers to perform sensitive image-guided surgery without line-of-sight restrictions. Accuracy of position measurement is important when guiding a precision instrument in a patient without a direct line of sight. Distortion may produce inaccurate position measurements and potential danger to a patient. Thus, a system that reduces inaccurate tracking measurements would be highly desirable. A system that minimizes the effect of distortion on position measurement would be highly desirable.

Thus, there is a need for a system and method for dynamic, improved calibration of instruments used in image-guided surgery and other navigation operations.

### BRIEF SUMMARY OF THE INVENTION

Certain embodiments of the present invention provide a method and system for improved tracking of an internal object, such as an implant or part of the anatomy, during an image-guided operation. In an embodiment, the method may include obtaining a plurality of identifying features or fiducials for an internal object from a plurality of projection views, obtaining a plurality of measurements for the internal object using the plurality of identifying features or fiducials, and forming a three-dimensional model of the internal object using the plurality of measurements from a plurality of projection views for use in tracking the internal object.

In an embodiment, the plurality of identifying features or fiducials may include an angle, an indentation, and/or a groove. The method may also include generating a mathematical model of the internal object. In addition, the three-dimensional model of the internal object may be compared with the mathematical model of the internal object to determine a variation. Tracking of the internal object may then be adjusted based on the variation. In an embodiment, the method may also include obtaining measurements of the internal object again after a deformation or a change in location or orientation of the internal object. In an embodiment, the method may also include correcting the projection views for distortions with a model, such as the Tsai model, for example.

In an embodiment, the system for improved tracking of an implant, includes an implant. The implant may be tracked with respect to a reference three-dimensional coordinate system. The system may also includes a plurality of fiducials placed on the implant and/or located using an image of the implant. The plurality of fiducials may enable measurement of the implant. The system may also includes a sensor for measuring the implant. The sensor may be capable of being positioned with respect to one or more of the fiducials for measurement of one or more locations on the implant. The system may also includes a tracking system for measuring one or more locations on the implant. The tracking system may use the measurements, the sensor, the plurality of fiducials and a plurality of projection views to form a three-dimensional model of the implant.

In an embodiment, the plurality of fiducials may include an angle, an indentation, a groove and/or an identifying feature. The sensor may be an electromagnetic sensor, an optical sensor, an ultrasound sensor, or an inertial position sensor. The sensor and tracking system may be sterile to maintain a sterile environment during calibration of the instrument. The tracking system may use a model to correct for distortions in the projection views, such as the Tsai projection model, for example. The tracking system may compare measurements from the instrument with a theoretical model for the instrument. In an embodiment, the system may also include a measurement frame for positioning the instrument for measurement. Measurements may include position and/or distance measurements, for example.

In an embodiment, the system includes an internal object. The internal object may be tracked with respect to a three-dimensional reference coordinate system during the image-guided operation. In an embodiment, the system also includes a plurality of projection views of the internal object. In an embodiment, the system also includes a plurality of images taken from the projection views of the internal object. The system may also include a plurality of identifying features or fiducials located in the images of the internal object. The system may also include a tracking system that measures one or more locations on the internal object. In an embodiment, the tracking system may form a three-dimensional internal object using the measurements, the images, the fiducials and/or identifying features, and a plurality of projection views.

In an embodiment, the identifying features or fiducials may include an angle, an indentation, and/or a groove. The tracking system may be sterile to maintain a sterile environment during tracking of the internal object. The tracking system may compare measurements from the instrument with a theoretical model for the instrument. The tracking system may use a model to correct for distortions in the projection views, such as the Tsai projection model, for example. In an embodiment, the system may also include an instrument for use in an image-guided operation. The instrument may be tracked with respect to the three-dimensional reference coordinate system and the internal object during an image-guided operation. In an embodiment, the system may also include a plurality of fiducials placed on the instrument. The plurality of fiducials may enable measuring of the instrument. In an embodiment, the system may also include a sensor for measuring the instrument. The sensor may be capable of being positioned with respect to one or more of said fiducials for measurement of one or more locations on the instrument by the tracking system. In an embodiment, the tracking system may measure one or more locations on the instrument using the sensor and the plurality of fiducials.

### BRIEF DESCRIPTION OF SEVERAL VIEWS OF THE DRAWINGS

FIG. 1 illustrates a flow diagram for a method for calibrating an instrument for use in surgical navigation or other image-guided operation in accordance with an embodiment of the present invention.

FIG. 2 illustrates an instrument calibration system used in accordance with an embodiment of the present invention.

FIG. 3 shows a model of an instrument used in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

In certain embodiments, image data sets of a patient or other object are obtained using an imaging system, such as a CT or MR imaging system. Several points may be identified on the patient (such as orbits around the nose, eyes, and/or ears). Matching points are then identified in the image data sets. Location data points in the image data sets are registered to actual locations on the patient in a reference coordinate frame. The images may then be used for surgical navigation.

A medical instrument may be analyzed with respect to the reference coordinate system for use in image-guided surgery and instrument tracking. Dimensions of the medical instrument may be determined within certain acceptable tolerances for medical applications. Reference or fiducial points are placed on the medical instrument. The reference points are then used to calibrate a section of the medical instrument for tracking.

A plurality of medical instruments may be calibrated for surgical navigation. For example, a reducer rod may be calibrated for surgical navigation. A reducer rod may be used in orthopedic surgery (trauma) for repairing a broken femur or other bone. For example, the reducer rod is used to align bone segments. The reducer rod runs down the center of the bone to wedge the bone segments back in place. The reducer rod may be used with surgical navigation to assist in the surgery. A flexible reducer rod can be more convenient for a doctor to use in manipulating the bone segments. However, a flexible rod tends to distort or bend more than a rigid rod. A stiff reducer is easier to calibrate but is less useful to a surgeon in an operation. Improved calibration of a flexible reducer rod improves ease of use and reliability in an operation.

Alternatively, a bone, any part of an anatomy, or an implant, rather than an instrument such as a reducer, may be calibrated. A position on or in the bone, any part of the anatomy, or the implant may be tracked during surgery. A flexible reducer rod may be used to provide a doctor with a sense of feel inside the patient without tracking the position of the rod with respect to the patient. Other instruments used in orthopedic and other surgery may also be calibrated for use in image-guided operations.

Parts of an anatomy may change before, during, or after surgery by bending, breaking, tearing, torqueing, splintering, warping, buckling, collapsing, puncturing, deflating, inflaming, and/or indenting, for example. Such part of the anatomy may include a bone, a bone segment, an organ, a tissue, a cartilage, a calcified structure, or any other part of a body, for example. Alternatively, an implant, rather than an instrument, may be calibrated. A position on or in the implant may be tracked during surgery. Such an implant may include a bone implant, organ implant, screw, staple, pacemaker, ball and socket, metal plate or any other foreign object inserted into the body, for example.

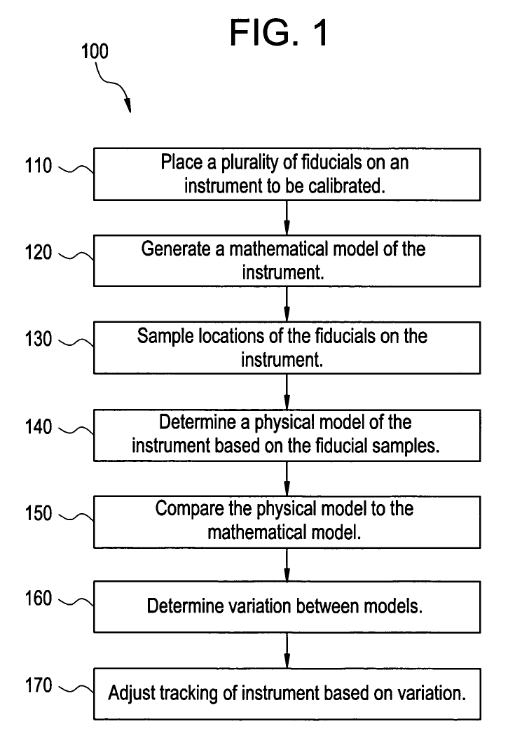

FIG. 1 illustrates a flow diagram for a method 100 for calibrating an instrument for use in surgical navigation or other image-guided operation in accordance with an embodiment of the present invention. Manufacture of tools may be crude and not very precise. However, using certain fiducials, tools may be accurately described for tracking in a reference coordinate system. First, at step 110, a plurality of fiducials are placed on an instrument to be calibrated. Fiducials may be placed on an instrument in regular or random intervals. For example, indentations may be made on the instrument, such as indentations along a side of a reducer rod or around a circumference of the rod. Alternatively, one or more grooves may be etched along a surface of the instrument, such as a continuous groove carved along a side of the reducer rod or around a circumference of the reducer rod. In an embodiment, identifying features of the instrument may be used as fiducials for calibration.

Next, at step 120, a computer model of the instrument is generated, representing points on the instrument in a tracking system reference frame. A computer-aided design (CAD) model or other mathematical or computer model may be formed to provide a two- or three-dimensional representation of the instrument in the reference coordinate system. The model represents an ideal or theoretical shape of the instrument. Then, at step 130, locations of the fiducials on the instrument are sampled. That is, a sensor or tracking device, such as an electromagnetic or optical tracking device, is used to measure actual positions on the instrument. For example, a tracker may be run through a groove carved on the instrument, and a plurality of positional data points may be obtained. Alternatively, for example, a tracker may be placed in a plurality of indentations on the instrument, and a plurality of positional data points may be obtained. At step 140, the plurality of fiducial samples may be used to determine a physical model of the instrument. The physical model reflects deformities or manufacturing flaws in the instrument.

Next, at step 150, the physical model is compared with the mathematical model. Sampled locations from the fiducials on the instrument are compared with reference coordinates from the computer-generated model of the instrument. Then, at step 160, variations in sampled locations from the actual model and sampled locations from the theoretical model are determined. A difference in a measured position and an ideal position on the instrument is determined. For example, an offset vector and error value (RMS or maximum error, for example) may be determined. The difference reflects imperfections or distortions in the instrument.

At step 170, variations in position measurements are used to adjust tracking of the instrument in image-guided operations. That is, the variation is used to adjust a tracking system used to track instrument position with respect to an image data set of a patient or other object. Adjustment improves tracking accuracy and enables instruments to be used in image-guided surgery or other image-guided operations despite defects. The instrument may be recalibrated between operations to readjust tracking accuracy. Additionally, the instrument may be calibrated with a sterile tracker during an operation, as the operation allows, in order to maintain tracking accuracy during the operation.

In another embodiment, an internal object, such as an implant or part of an anatomy, instead of an instrument, may be tracked during surgical navigation or other image-guided operation. Using certain fiducials, implants may be accurately described for tracking in a reference coordinate system. Using identifying features, both implants and parts of the anatomy may be accurately described for tracking in a reference coordinate system. The identifying features may include an angle, an indentation, and/or a groove, for example.

First, a plurality of fiducials may be placed on an implant to be calibrated. Fiducials may be placed on an implant in regular and/or random intervals. For example, indentations may be made on the implant, such as indentations along a side of a femur implant or around a circumference of the implant. Alternatively, one or more grooves may be etched along a surface of the implant, such as a continuous groove carved along a side of the femur implant or around a circumference of the femur implant. In an embodiment, identifying features of the implant may be used as fiducials for calibration.

Alternatively, preexisting identifying features on implants or parts of an anatomy may perform the same function as fiducials in identifying, measuring and tracking an implant or a part of the anatomy. Identifying features may include a groove, indentation, curve, or any other aspect of the implant or part of the anatomy that makes the implant or part of the anatomy distinguishable and thus identifiable. For example, in an embodiment, identifying features of a femur may include Greater Trochanter, Medial Epicondyle, Lateral Epicondyle, Adductor Tubercle, for example. Both an implant and parts of an anatomy are internal objects to a body.

A computer model of an internal object may be generated, representing points on the internal objects in a tracking system reference frame. A computer-aided design (CAD) model or other mathematical or computer model may be formed to provide a two- or three-dimensional representation of the internal object in the reference coordinate system. The model represents an ideal or theoretical shape of the internal object. Locations of the fiducials or identifying features on the internal object may be sampled.

In an embodiment, a sensor or tracking device, such as an electromagnetic or optical tracking device, may be used to measure actual positions on an internal object, such as an implant. For example, a tracker may be run through a groove carved on the implant, and a plurality of positional data points may be obtained. Alternatively, for example, a tracker may be placed in a plurality of indentations on the implant, and a plurality of positional data points may be obtained. The plurality of fiducial samples may be used to determine a three-dimensional physical model of the implant.

Alternatively, a plurality of identifying features on an internal object may be identified in images taken of the internal object. Obtaining a plurality of images from a plurality of projection views allows a three-dimensional physical model of the internal object to be created. The physical model reflects deformities or manufacturing flaws in an implant. The physical model reflects changes or abnormalities in a part of an anatomy. A model, such as the Tsai model, for example, may be used to correct for distortion in the images.

The physical model is compared with the mathematical model. Sampled locations from the fiducials or identifying features on the internal object are compared with reference coordinates from the computer-generated model of the internal object. Variations in sampled locations from the actual model and sampled locations from the theoretical model are determined. A difference in a measured position and an ideal position on the internal object is determined. For example, an offset vector and error value (RMS or maximum error, for example) may be determined. The difference reflects imperfections or distortions in the internal object. Alternatively, for example a quaternion or affine registration matrix representation an offset may be used with an error value to determine a difference in position as well as relative rotations. In an embodiment, a space distortion tensor may be used to represent bending, shrinkage, and/or expansion of the internal object, for example.

Variations in position measurements may be used to adjust tracking of the internal object in image-guided operations. That is, the variation may be used to adjust a tracking system used to track the position of an instrument position with respect to an internal object. Adjustment improves tracking accuracy and enables an instrument to be used in image-guided surgery or other image-guided operations despite defects, variations, or changes in surrounding internal objects. The variation may also be used to obtain a better understanding of how the internal object has been deformed or moved. The internal object may be tracked between operations to readjust tracking accuracy. Additionally, the internal object may be tracked with a sterile tracker during an operation, as the operation allows, in order to maintain tracking accuracy during the operation.

In another embodiment, points may be sampled from an acquired image of the instrument. One or more images of the instrument may be obtained. Locations or fiducials on the instrument may be sampled in the image(s) and correlated to generate a two- or three-dimensional model of the instrument. The model may then be compared with a theoretical model of the instrument to identify and compensate for distortions or imperfections in the instrument.

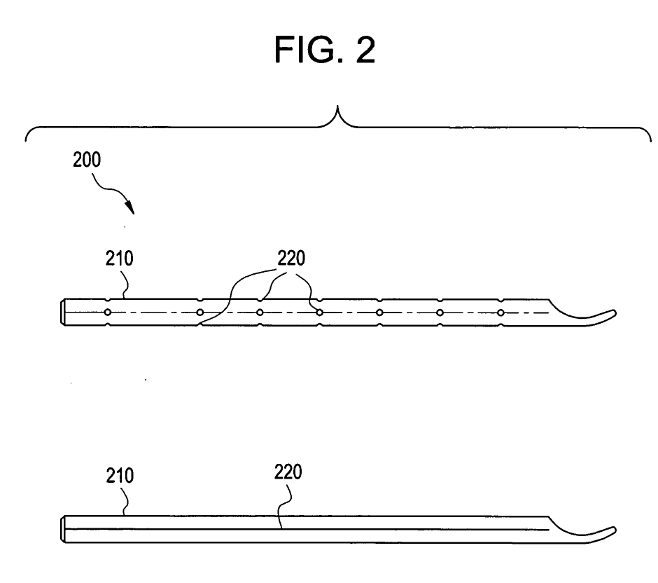

FIG. 2 illustrates an instrument calibration system 200 used in accordance with an embodiment of the present invention. For illustration purposes only, the instrument is described as a reducer rod but may be any of a variety of instruments or implants used in medical procedures or other image-guided or tracking applications. The instrument calibration system 200 includes an instrument 210 and a plurality of fiducials 220. The system also includes a sensor 230 and a measurement frame 240, which are not shown in FIG. 2.

The fiducials 220 are markers or indications along the instrument 210 that may be used to identify or measure positions along the surface of the instrument 210. The fiducials 220 may be spaced at regular intervals along the instrument 210 and/or at irregular intervals along the instrument 210. A positional map or model may be created based on measurements of the fiducials 220 to define the shape and dimension of the instrument 210 in a reference coordinate system. The map or model may then be used with an image data set for image-guided tracking operations, such as surgical procedures. The fiducials 220, for example, may be indentations, grooves and/or identifying features in the instrument 210. That is, the fiducials 220 may be holes or indentations along the outer surface of the instrument 210 or through the surface of the instrument 210, as shown in the top portion of FIG. 2. Fiducials 220 may also be grooves or lines etched or cut along the surface of the instrument 210, for example (as shown in the bottom portion of FIG. 2). Grooves in the instrument 210 may be cut vertically, horizontally, diagonally, and/or around a circumference of the instrument 210. Features or components of the instrument 210 may also be used to identify locations on the instrument 210.

The sensor 230 enables measurements to be taken for various points on the instrument 210. The sensor 230 is placed on or in the fiducials 220. The sensor 230 may be an electromagnetic tracker, an optical sensor, an inertial position sensor, or an ultrasound sensor, for example. The sensor 230 may be a transmitter transmitting a signal to a receiver which determines a relative location of the sensor 230. The sensor 230 may also be a receiver receiving a signal from a transmitter and determining a relative location of the sensor 230 in a reference coordinate system. The sensor 230 is positioned in one or more of the plurality of fiducials 220 to obtain a plurality of position measurements along the instrument in the reference coordinate system.

For example, an electromagnetic sensor with a spherical tip may be placed in each of a plurality of indentations on a reducer rod to obtain positional measurements along the reducer rod in a reference coordinate system. A sensor with a narrow tip, for example, may be placed in one or more fiducial grooves along a reducer rod, for example, and moved along the groove(s) to obtain positional measurements in a reference coordinate system. In an embodiment, a plurality of sensors 230 may be used with a plurality of fiducials 220 to simultaneously obtain a plurality of position measurements.

The measurement frame 240 is used to position the instrument 210 such that the sensor 230 may be used to obtain positional measurements of the fiducials 220. The measurement frame 240 serves to position or immobilize the instrument 210 in a reference frame for measurement using the sensor 230 and a measurement system, such as an electromagnetic or optical tracking system. For example, the measurement frame 240 may be a positioning arm or a board and locking mechanism. The measurement frame 240 may also allow the instrument 210 to be moved (spun or rotated, for example) while the sensor 230 is positioned in the fiducials 220 to obtain position measurements.

In operation, the instrument 210 is placed in the measurement frame 240. The measurement frame 240 and sensor 230 may be sterile to protect sterility of the instrument 210 and of the operating environment. For example, an end of a reducer rod may be placed in a dimple or socket in the measurement frame 240. The sensor 230 is placed in one of the fiducials 220. The sensor 230 is used with a tracking or measurement system, such as an electromagnetic or optical transmitter/receiver, to determine a position of the sensor 230 with respect to the measurement frame 240. A position of a fiducial 220 with respect to the measurement frame 240 and/or a position of a fiducial 220 with respect to another fiducial 220 are used to determine relative position and dimension of the instrument 210. In an alternative embodiment, the instrument 210 in the instrument calibration system 200 may be an implant.

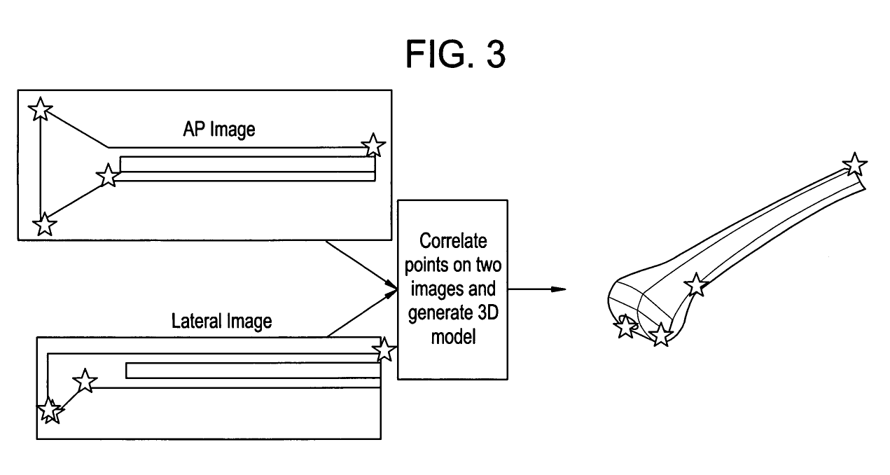

In an alternative embodiment, fiducials 220, identifying features or locations may be sampled from images acquired of a part of an anatomy, the instrument 210, or an implant without the use of a sensor. For example, as shown in FIG. 3, fiducials 220 identified on two images may be correlated to generate a three-dimensional model of a part of the anatomy, implant or instrument 210. Positions of fiducials 220 in the model are used to determine relative position and dimension of the part of the anatomy, implant or instrument 210 in a reference frame.

Identifying features of an internal object, such as a part of an anatomy or an implant, may be treated as fiducials 220. Identifying features are indications along the internal object that may be used to identify or measure positions along the surface of the internal object or to identify the internal object itself. The identifying features, for example, may be indentations, grooves and/or curves on the internal object. A theoretical model of an internal object may aid in the locating and determining which features to designate as identifying features. A positional map or model may be created based on measurements of the identifying features to define the shape and dimension of the internal object in a reference coordinate system. The map or model may then be used with an image data set for image-guided tracking operations, such as surgical procedures.

A plurality of projection views may be necessary to properly generate a three-dimensional image of the internal object in order to circumvent or manipulate the internal object in an image-guided surgery. Increasing the number of identifying features, fiducials, projection views and images taken of a part of the internal object increases the accuracy with which the three-dimensional model reflects the part of the internal object, implant or instrument 210. Once the three-dimensional model is made, the internal object may be tracked with an image from a projection view of the object. The fiducials or identifying features in the image may determine the orientation of the internal object and whether a change in the object has occurred.

In an embodiment, an internal object may be recalibrated during an operation. Recalibration may include obtaining a plurality of images from a plurality of projection views, recognizing identifying features or fiducials in the images, taking measurements of the identifying features, and using the measurements to form a three-dimensional physical model of the internal object.

In an embodiment, the three-dimensional physical model may be used to properly align the internal object. There is a plurality of applications of tracking an internal object with a three-dimensional model using two-dimensional images. Repairing a fractured femur, for example, is just one of them. The fractured femur needs to be aligned along an axis to set the broken leg and allow the leg to heal properly. The three-dimensional model would enable better tracking and alignment of the bone segments of the fractured femur, especially during image-guided surgery.

Measuring a plurality of fiducials 220 improves accuracy and reduces error in a model of the instrument 210. In an embodiment, obtaining a plurality of position measurements using a fiducial 220 (such as by rotating the instrument with the sensor 230 in a fiducial 220) also improves positional accuracy. Using multiple fiducials 220 along a surface of the instrument 210 allows deformities or imperfections in the instrument 210 to be more accurately modeled and accommodated during image-guided surgery. Repetition in calibration measurements may also improve model and tracking accuracy. A model formed from fiducial 220 measurements may be used with images obtained of an object, such as a patient, to perform an image-guided task, such as image-guided surgery.

In an embodiment, a feedback mechanism is used during surgical navigation to determine accuracy of tracking of an instrument. Comparison of tracked versus actual instrument position may be quickly checked during a procedure. If positional accuracy is within acceptable limits, then the procedure may continue with the instrument. If positional accuracy is outside an acceptable tolerance or if the instrument is incapable of tracking due to a deformity, then the instrument may be recalibrated quickly during the procedure. Then the instrument may continue to be used in the procedure.

Certain embodiments use closed form registration to calibrate deformed instruments in a surgical navigation system. Using closed form registration of an instrument, a solution is overdetermined using multiple data points. That is, the geometry of an instrument may be described specifically. A CAD model of the instrument, such as a reducer rod, is created. Several fixed points on the rod are sampled. A mathematical model of where the sampled points should theoretically be located is generated using the CAD model. Using the sampled coordinates and the ideal coordinates, a solution for the position of the sampled points in a reference coordinate frame is determined.

In an embodiment, the reference frame may be defined using a hole or indentation in a measuring surface. A sensor, such as an electromagnetic sensor, or other tracking device is placed on one end of an instrument to be calibrated, such as a reducer rod. The other end of the rod is placed in the hole or indentation. The rod is spun around in the hole, and position is measured using the sensor or other tracking device. A distance between the sensor and the hole is known. An angle of the sensor with respect to the hole changes as the rod is rotated. Measurement may be repeated with the sensor located at various points on the instrument. Using the distance and angle measurements, a closed form solution may be calculated for the rod.

Thus, certain embodiments provide a system and method for closed form registration of an instrument with respect to a tracking system reference frame. Certain embodiments allow an imperfect instrument to be used with image-guided operations with improved safety and accuracy. Certain embodiments provide dynamic calibration of an instrument to allow continued use and tracking of the instrument despite deformity or imperfection. Sampling a plurality of points on an instrument allows improved indication of a problem with an instrument and a broader statistical base for calibration quality. An increased number of data points and/or redundant data points provide an improved instrument model and improved tracking for image-guided navigation.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for improved tracking of an internal object, said method comprising:
obtaining a plurality of identifying features for said internal object from a plurality of projection views, wherein said internal object comprises at least one of an implant and a part of an anatomy;
obtaining a plurality of measurements for said internal object from said plurality of projection views using said plurality of identifying features; and
forming a three-dimensional model of said internal object using said plurality of measurements for use in tracking said internal object.

2. The method of claim 1, wherein said identifying features comprise at least one of an angle, an indentation, and a groove.

3. The method of claim 1, further comprising generating a mathematical model of said internal object.

4. The method of claim 3, further comprising comparing said three-dimensional model and mathematical model of said internal object to determine a variation.

5. The method of claim 1, further comprising obtaining a plurality of measurements for said internal object after at least one a deformation, a change in location and a change in orientation of said internal object.

6. The method of claim 1, further comprising correcting said projection views for distortions with a model.

7. A system for improved tracking of an implant, said system comprising:
an implant, said implant tracked with respect to a reference three-dimensional coordinate system;
at least one of a plurality of fiducials placed on said implant and a plurality of fiducials located using an image of said implant, wherein both of said pluralities of fiducials enable measurement of said implant;
a sensor for measuring said implant, said sensor capable of being positioned with respect to one or more of said fiducials for measurement of one or more locations on said implant; and
a tracking system for measuring one or more locations on said implant using said sensor and said plurality of fiducials and a plurality of projection views to form a three-dimensional model of said implant.

8. The system of claim 7, wherein both of said pluralities of fiducials comprise at least one of an angle, an indentation, a groove, and an identifying feature.

9. The system of claim 7, wherein said sensor comprises at least one of an electromagnetic sensor, an optical sensor, an ultrasound sensor, and an inertial position sensor.

10. The system of claim 7, wherein said sensor and said tracking system comprise a sterile sensor and sterile tracking system.

11. The system of claim 7, wherein said tracking system uses a model to correct for distortions in said projection views.

12. The system of claim 7, wherein said tracking system compares measurements from said implant with a theoretical model for said implant.

13. The system of claim 7, further comprising a measurement frame for positioning said implant for measurement.

14. A system for improved tracking of an internal object, said system comprising:
an internal object, wherein said internal object comprises at least one of an implant and a part of an anatomy, said internal object tracked with respect to a reference three-dimensional coordinate system;
a plurality of projection views of said internal object;
a plurality of images taken from said projection views of said internal object;
a plurality of at least one of identifying features and fiducials on said internal object identified in said images, said plurality of at least one of identifying features and fiducials enabling measurement of said internal object;
a tracking system for measuring one or more locations on said internal object using said images, said plurality of at least one of identifying features and fiducials and a plurality of projection views to form a three-dimensional model of said internal object.

15. The system of claim 14, wherein said plurality of at least one of identifying features and fiducials comprise at least one of an angle, an indentation, and a groove.

16. The system of claim 14, wherein said tracking system comprises a sterile tracking system.

17. The system of claim 14, wherein said tracking system compares measurements from said internal object with a theoretical model for said internal object.

18. The system of claim 14, wherein said tracking system uses a model to correct for distortions in said projection views.

19. The system of claim 18, wherein said model is the Tsai projection model.

20. The system of claim 14, further comprising:
an instrument for use in an image-guided operation, said instrument tracked with respect to said three-dimensional reference coordinate system and said internal object during said image-guided operation;
a plurality of fiducials placed on said instrument, said plurality of fiducials enabling measurement of said instrument; and
a sensor for measuring said instrument, said sensor capable of being positioned with respect to one or more of said fiducials for measurement of one or more locations on said instrument by said tracking system, wherein said tracking system measures one or more locations on said instrument using said sensor and said plurality of fiducials.
